(19) United States
(12) Patent Application Publication
(10) Pub. No.: US 2008/0034454 A1
(43) Pub. Date: Feb. 7, 2008

(54) Artificial seed

(76) Inventor: Asaoka Keiichiro, Atsugi City (JP)

Correspondence Address: BUHLER ASSOCIATES;BUHLER, KIRK A., 1101 CALIFORNIA AVE., SUITE 208, CORONA, CA, 92881, US

(21) Appl. No.: 11/541,126

(22) Filed: Oct. 2, 2006

(30) Foreign Application Priority Data
Aug. 1, 2006 (JP) PCT/JP2006/315207

(51) Int. Cl. A01H 5/00

(52) U.S. Cl. 800/298

(57) ABSTRACT

The present invention provides the artificial seed which is made from plant cell with simple process and which can tremendously raise the plant without changing its gene tissues.

The artificial seed which is made from the plant cells at growing point by trimming the growing part, subdividing the part, adding organic acid solution of pH 5-7 and mixing with porous particles.

### CROSS REFERENCE TO RELATED APPLICATION

This application claims priority to Japan application number PCT/JP2006/315207 filed on Aug. 1, 2006.

### DESCRIPTION

1. Field of the Invention

The present Invention is related to the artificial seed which is made from cells at the growing point of a plant.

2. Background of the Invention

Most of plants make their seeds, and the seeds germinate to raise their new plants. Various way of raising plant bodies without depending on germination of seed is well known. For example, branch cuttings or bud cuttings, is a way of raising plant by taking a growing young branch of plant. Another way to raise a plant body is to use a branch of plant and germinate roots by surrounding the cut end with moisture and peeling off its bark, or layering, which is a way to separate branch of plant after having it germinate roots.

A cloning technology for plant, is another way of increasing efficiency to culture adventitious embryo by adding organic acid to the nutrient medium, at forming embryo tissue from somatic cell tissue of plant which forms adventitious embryo with regeneration of somatic cell tissue from the derivative cell in the nutrient culture medium is known (Refer to the patent literature 1).

Current conventional plant cloning technology requires a large facility and extensive labor in order to form adventitious embryo because the forming of adventitious embryo is done with cell culture in Nutrient Medium.

Artificial seed, which is able to be reserved at drying condition, made from adventitious embryo being wrapped with soft capsule of three layers is also known. (For example, refer to patent literature 2)

Patent literature 1: (Japanese) Patent publication #2688621

Patent literature 2: (Japanese) Patent Publication #2002-360665

### BRIEF SUMMARY OF THE INVENTION

It is an object of the artificial seed technology to grow a plant that does not grow its seed, or in the case where the season is not at the time of seed born or even in case that its branch cuttings, bud cuttings or layering is difficult to raise the plant, can raise the plant body without changing its gene tissue at all.

It is further an object of the artificial seed to provide artificial seeds which can tremendously raise the plant by reforming its plant cell to seed with a simple process without changing its gene tissue.

### Means to Achieve the Theme

In order to achieve the above mentioned theme, the present invention provides the artificial seed which is reformed from cells at growing point by trimming the growing part of plant, subdividing the part, adding organic acid solution of pH 5-7, and mixing with porous particles.

It is further an object of the artificial seed of wherein the artificial seed is added with adsorption promoting bacteria to promote adsorption of organic acid into the above mentioned cells at growing point.

It is further an object of the artificial seed wherein the aforementioned adsorption promoting bacteria is aspergillus Awamori (black aspergillus oryzae) or aspergillus kawachi (malted rice aspergillus).

It is further an object of artificial seed wherein the aforementioned organic acid is citric acid.

It is further an object of the artificial seed wherein the aforementioned porous particles are Coral Sand or Coral Particle which is treated with being desalted and then dried.

It is further an object of the artificial seed wherein the aforementioned cells at growing point are added with magnesium solution.

It is further an object of the artificial seed wherein the aforementioned cells at growing point are added with sodium solution.

It is further an object of the artificial seed wherein the aforementioned cells at growing point are added with glucose solution.

### THE EFFECT OF THE INVENTION

The artificial seed technology provides enhanced germination of cells at growing point by the process of trimming the growing part of plant, subdividing the part, adding organic acid solution of pH 5-7 which is the specific pH of the plant, mixing with porous particles.

It is an object of the making of artificial seeds in a very simple method by mixing the cells at growing point with porous particles which keeps the cells at growing point at dry condition and reforms the cells to artificial seed.

It is further an object of the artificial seed to effect the adsorption-promoting bacteria which enhance the germination of cells at growing point of artificial seed by having the cells at growing point adsorb organic acid effectively into the cells at growing point.

It is further an object of the artificial seed to effect the aforementioned adsorption-promoting bacteria which enhance the adsorption of organic acid solution effectively into the cells at growing point of artificial seed wherein the bacteria is aspergillus awamori or aspergillus kawachi.

It is further an object of the artificial seed to affect the aforementioned organic acid which enhances the germination of cells at growing point of artificial seed where the organic acid is citric acid.

It is further an object of the artificial seed where the technology is related to the effect of porous particles which keep the dry condition of artificial seed. The aforementioned porous particles can be easily obtained by treating Coral Sand or Coral particles being desalted and dried.

It is further an object of the artificial seed where the effect of adding magnesium to the cells at growing point which enhance the germination of artificial seed that gives a signal to start germination.

It is further an object of the artificial seed where the effect of adding sodium solution to the cells at growing point, which secures the start-signal of germination of the artificial seeds is integrated with the effect of magnesium solution.

It is further an object of the artificial seed where the effect of glucose which enhances the growth of cells at growing point by providing nutriment after being germinated to the aforementioned cells at growing point of artificial seed.

Various objects, features, aspects, and advantages of the present invention and method will become more apparent from the following detailed description of preferred embodiments of the invention, along with the accompanying drawings in which like numerals represent like components.

### DETAILED DESCRIPTION

### The Preferred Method for Embodiment

The artificial seed which is related to the present invention is made by the process of trimming growing parts of plant containing the cells at growing point, subdividing the growing parts, adding organic acid solution of pH 5-7 to the cells at growing point and mixing with porous particles.

### Embodiment of the Present Invention

The growing point of plant is at the tip part of stem or root of which cells is lively dividing and generating its new tissues of plant.

One of the embodiments of the present invention used “portulaca oleracea” (Japanese:“Suberihiyu”, a plant of “Suberihiyu” family).

About 10 gram of growing point of portulaca oleracea was trimmed and was dispersed in water by blender by which the cells at growing point were subdivided into cell unit.

In case of the above mentioned embodiment, the organic acid solution was added to promote the cells at growing point to become seeds and to germinate, and 5 gram of citric acid was dissolved in water to make solution of pH6.

Together with the citric acid solution of pH 6, the cells at growing point were added with aspergillus awamoi as adsorbent-promoting bacteria which enhances the adsorption of organic acid into the cells.

It is possible to use aspergillus kawachi in stead of aspergillus awamoi as the adsorption-promoting bacteria.

The adsorption-promoting bacteria can enhance the germination of cells at growing point by having the cells adsorb organic acid such as citric acid effectively.

Furthermore, the pH of organic acid solution is preferably to be adjusted as pH 5-7 which is matching to the pH of plant for efficient adsorption.

The quantity of organic acid to be added is preferably at the ratio of 1:(0.2-1.0) by weight of the cells at growing point and citric acid (2-10 gram, in case of the above mentioned embodiment) in order for the cells at growing point to adsorb the organic acid effectively.

In case of the above mentioned embodiment, coral sand and coral particles, which were desalted and dried, were used as the porous particles.

The porous particle of coral sand and particles were well washed to remove salt and were dried up by heating.

With this pretreatment, the porous particles are not only kept dry but also kept sterilized.

The coral of large size is preferably crushed into sand-size.

The artificial seed of the present invention was formed with the cells at growing point which were added with the organic acid and then mixed with the above mentioned porous particles.

By this process, the cells at growing point became artificial seed which can be maintained at a certain drying condition that regulates the division of cells.

As the porous particle material, the similar porous construction material can be used replacing coral sand or coral particles.

In case of the above mentioned embodiment, the cells at growing point were added with magnesium solution and sodium solution at the seed-time.

The magnesium solution is for the purpose of giving germination-start signal to the cells at growing point of artificial seed, and the sodium solution is for the purpose of enhancing the function of magnesium.

The amount of magnesium and sodium solution was smaller quantity compared with the aforementioned organic acid solution.

Furthermore, the cells at growing point were added with glucose solution in order to provide nutriment to the artificial seed at the seeding time and after the germination.

In the abovementioned embodiment, glucose solution of 10% concentration was sprayed over the artificial seed.

The concentration of glucose solution is preferably 7-13% with which the glucose is easily penetrate into the cell shells of plant.

In case of the above-mentioned embodiment, the artificial seed was sowed on coral sand for germination.

The germination temperature of portulaca oleracea is 22-24 Deg. C. 500 to 1,000 spouts were germinated from the artificial seed which had been made from about 10 gram of growing point of portulaca oleracea.

Thus, specific embodiments of producing artificial seed have been disclosed. It should be apparent, however, to those skilled in the art that many more modifications besides those described are possible without departing from the inventive concepts herein. The inventive subject matter, therefore, is not to be restricted except in the spirit of the appended claims.

What is claimed is:

1. Artificial seed made from cells comprising:
artificial seed which is made from cells at growing point of plant by trimming the growing part from the plant, subdividing the part, adding organic acid of pH 5-7, and mixing with porous particles.

2. The artificial seed from claim 1 wherein the artificial seed is added with absorption-promotion bacteria to enhance the absorption of organic acid to the above-mentioned cells at growing point.

3. The artificial seed from claim 2 wherein the aforementioned adsorption-promoting bacteria is aspergillus awamori.

4. The artificial seed from claim 2 wherein the aforementioned adsorption-promoting bacteria is aspergillus kawachi.

5. The artificial seed from claim 1 wherein the aforementioned organic acid is citric acid.

6. The artificial seed from claim 1 wherein the aforementioned porous particles are coral sand or coral particles.

7. The coral or sand particles from claim 6 wherein the particles are desalted and dried.

8. The artificial seed from claim 1 wherein the aforementioned cells at growing point are added with magnesium solution.

9. The artificial seed from claim 8 wherein the aforementioned cells at growing point are added with sodium solution.

10. The artificial seed from claim 1 wherein the aforementioned cells at growing point are added with glucose solution.
